(19) United States
(12) Patent Application Publication
(10) Pub. No.: US 2016/0263116 A1
(43) Pub. Date: Sep. 15, 2016

(54) COMBINATIONS

(71) Applicant: GlaxoSmithKline LLC, Wilmington, DE (US)

(72) Inventors: Kurt Robert AUGER, Collegeville, PA (US); Vijay Gopal Reddy PEDDAREDDIGARI, Philadelphia, PA (US)

(21) Appl. No.: 14/680,416

(22) PCT Filed: Oct. 10, 2013
(86) PCT No.: PCT/US13/64260

§ 371 Date: Apr. 7, 2015

Related U.S. Application Data
(60) Provisional application No. 61/712,869, filed on Oct. 12, 2012.
(60) Provisional application No. 61/833,561, filed on Jun. 11, 2013.

(51) Int. Cl. A61K 31/519; A61K 31/4439

(52) U.S. Cl. CPC A61K 31/519; A61K 31/4439

(57) ABSTRACT

The present invention relates to a method of treating cancer in a human and to pharmaceutical combinations useful in such treatment. In particular, the method relates to a cancer treatment method that includes administering 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, to a human in need thereof.

### FIELD OF THE INVENTION

The present invention relates to a method of treating cancer in a mammal and to combinations useful in such treatment. In particular, the present invention relates to a novel combination comprising one Focal Adhesion Kinase (FAK) inhibitor: 2-[(5-Chloro-2-{[3-methyl-1-(1-methyl ethyl)-1H-pyrazol-5-yl]amino}-4-pyridinyl)amino]-N-(methyloxy)benzamide, or a pharmaceutically acceptable salt thereof, and one Mitogen-activated protein (MAP) kinase/extracellular signal-regulated kinase (ERK) kinase (hereinafter referred to as MEK) inhibitor: N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, pharmaceutical compositions comprising the same, and methods of using such combinations in the treatment of cancer.

### BACKGROUND OF THE INVENTION

Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

Tyrosine kinases play an important role in the regulation of many cell processes including cell proliferation, cell survival, and cell migration. It is known that certain tyrosine kinases become activated by mutation or are abnormally expressed in many human cancers. For example, the epidermal growth factor receptor (EGFR) is found mutated and/or overexpressed in breast, lung, brain, squamous cell, gastric, and other human cancers. Selective inhibitors of the tyrosine kinase activity of EGFR have been shown to be of clinical value in treatment of cancers with mutated and/or overexpressed EGFR. Thus, selective inhibitors of particular tyrosine kinases are useful in the treatment of proliferative diseases such as cancer.

FAK (encoded by the gene PTK2) is a non-receptor tyrosine kinase that integrates signals from integrins and growth factor receptors. FAK has been reported to play a role in the regulation of cell survival, growth, adhesion, migration, and invasion (McLean et al 2005, Nat Rev Cancer 5:505-515). Furthermore, FAK is regulated and activated by phosphorylation on multiple tyrosine residues. Overexpression of FAK mRNA and/or protein has been documented in many solid human tumors, including but not limited to, cancers of the breast, colon, (hyroid, lung, ovary, and prostate; but also including cancers of hematological origin, including but not limited to leukemia such as acute myeloid leukemia (AML). (Owens et al. 1995, Cancer Research 55: 2752-2755; Agochiya et al. 1999, Oncogene 18: 5646-5653; Gabarro-Niecko et al. 2003, Cancer Metastasis Rev. 22:359-374; Recher et al. 2004, Cancer Research 64:3191-3197; Zhao and Guan, 28:35-49, 2009, Cancer Metastasis Rev.). More significantly, there is evidence that phosphorylated FAK is increased in malignant compared to normal tissues (Grisaru-Granovsky et al. 2005, Int. J. Cancer 113: 372-378) and could represent a prognostic marker of metastasis FAK activity is clearly implicated in advanced and metastatic human cancer (Zhao and Guan, 28:35-49, 2009, Cancer Metastasis Rev.).

MEK is known to be involved in the regulation of cell proliferation as a kinase that mediates Raf-MEK-ERK signal transduction pathway, and the Raf family (B-Raf, C-Raf etc.) activates the MEK family (MEK-1, MEK-2 etc.) and the MEK family activates the ERK family (ERK-1 and ERK-2).

Activation of Raf-MEK-ERK signal transduction pathway in cancer, particularly colorectal cancer, pancreatic cancer, lung cancer, breast cancer and the like, has been frequently observed.

In addition, since the signals produced by signal molecules such as growth factors, cytokines and the like converge to the activation of MEK-ERK, inhibitors of these functions are considered to more effectively suppress Raf-MEK-ERK signal transduction than suppression of the function of upstream kinases such as RTK, and Raf.

Moreover, it is also known that a compound having MEK inhibitory activity effectively induces inhibition of ERK1/2 activity and suppression of cell proliferation (The Journal of Biological Chemistry, vol. 276, No. 4, pp. 2686-2692, 2001), and the compound is expected to show effects on diseases caused by undesirable cell proliferation, such as tumor genesis and/or cancer.

It would be useful to provide a novel therapy which provides more effective and/or enhanced treatment of an individual suffering the effects of cancer.

### SUMMARY OF THE INVENTION

One embodiment of this invention provides a combination comprising:

(i) a compound of Structure (I):

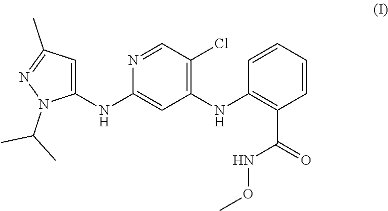 [SMILES: CONC(=O)c1ccccc1Nc1cc(Nc2cc(C)nn2C(C)C)ncc1Cl]

or a pharmaceutically acceptable salt thereof; and

(ii) a compound of Structure (II):

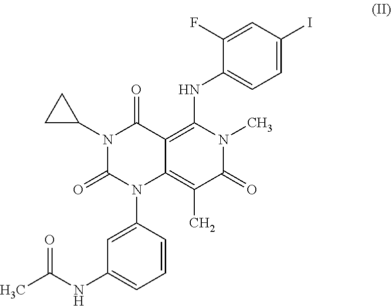 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof.

One embodiment of this invention provides a method of treating cancer in a human in need thereof which comprises the in vivo administration of a therapeutically effective amount of a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, to such human.

One embodiment of this invention provides a method of treating cancer in a human in need thereof which comprises the in vivo administration of a therapeutically effective amount of a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, to such human, wherein the combination is administered within a specified period, and wherein the combination is administered for a duration of time.

One embodiment of this invention provides a method of treating cancer in a human in need thereof which comprises the in vivo administration of a therapeutically effective amount of a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, to such human, wherein the compounds of the combination are administered sequentially.

### BRIEF DESCRIPTION OF THE DRAWINGS

FIG. 1 depicts the dose response curves of cell growth inhibition by Compound A. Compound B or a combination of Compound A and Compound B on the growth of Mero-82 cells.

FIG. 2 depicts the dose response curves of cell growth inhibition by Compound A, Compound B or a combination of Compound A and Compound B on the growth of NCI-H2052 cells.

FIG. 3 depicts the dose response curves of cell growth inhibition by Compound A. Compound B or a combination of Compound A and Compound B on the growth of NO36 cells.

FIG. 4 depicts the net change in the cell population for Compound A and Compound B as single agents or in combination on the growth of multiple mesothelioma cell lines.

FIG. 5 depicts the death EC₅₀ (dEC₅₀) values for Compound A dEC₅₀ values compared with the dEC₅₀ values from the Compound A and Compound B combination in multiple mesothelioma cell lines.

FIG. 6 depicts the death EC₅₀ (dEC₅₀) values for Compound B dEC₅₀ values compared with the dEC₅₀ values from the Compound A and Compound B combination in multiple mesothelioma cell lines.

### DETAILED DESCRIPTION OF THE INVENTION

The present invention relates to a method of treating cancer using combinations that exhibit antiproliferative activity. Suitably, the method relates to treating cancer by the co-administration of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, suitably the hydrochloride salt (hereinafter Compound A, or a pharmaceutically acceptable salt thereof), which compound is represented by Structure (I):
and

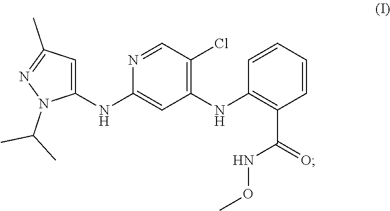 [SMILES: CONC(=O)c1ccccc1Nc1cc(Nc2cc(C)nn2C(C)C)ncc1Cl]

N-{3-[3-cyclopropyl-5-(2-fluoro-4-iodo-phenylamino)-6,8-dimethy-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof (hereinafter Compound B or a pharmaceutically acceptable salt or solvate, suitably the dimethyl sulfoxide solvate, thereof), which compound is represented by Structure (II):

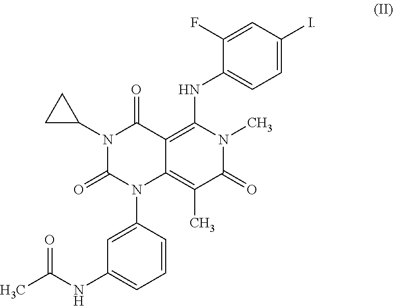 [SMILES: CC(=O)Nc1cccc(-n2c(=O)n(C3CC3)c(=O)c3c(Nc4ccc(I)cc4F)n(C)c(=O)c(C)c32)c1]

Compound A is disclosed and claimed, along with pharmaceutically acceptable salts thereof, as being useful as an inhibitor of FAK activity, particularly in treatment of cancer, in International Application No. PCT/US09/62163, having an International filing date of Oct. 27, 2009, International Publication Number WO 2010/062578 and an International Publication date of Jun. 3, 2010, the entire disclosure of which is hereby incorporated by reference, Compound A is the compound of Example 41a or 41b. Compound A, and the hydrochloride salt thereof can be prepared as described in International Application No. PCT/US09/62163.

Compound B is disclosed and claimed, along with pharmaceutically acceptable salts and solvates thereof, as being useful as an inhibitor of MEK activity, particularly in treatment of cancer, in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference, Compound B is the compound of Example 4-1. Compound B can be prepared as described in International Application No. PCT/JP2005/011082. Compound B can be prepared as described in United States Patent Publication No. US 2006/0014768, Published Jan. 19, 2006, the entire disclosure of which is hereby incorporated by reference.

Suitably, Compound B is in the form of a dimethyl sulfoxide solvate. Suitably, Compound B is in the form of a sodium salt. Suitably, Compound B is in the form of a solvate selected from: hydrate, acetic acid, ethanol, nitromethane, chlorobenzene, 1-pentanci, isopropyl alcohol, ethylene glycol and 3-methyl-1-butanol. These solvates and salt forms can be prepared by one of skill in the art from the description in International Application No. PCT/JP2005/011082 or United States Patent Publication No. US 2006/0014768.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable salts thereof, and Compound B, and pharmaceutically acceptable salt or solvate thereof.

The compounds of the invention may form a solvate which is understood to be a complex of variable stoichiometry formed by a solute (in this invention. Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Suitably the solvent used is water or dimethyl sulfoxide.

The pharmaceutically acceptable salts of the compounds of the invention are readily prepared by those of skill in the art.

Also, contemplated herein is a method of treating cancer using a combination of the invention where Compound A, or a pharmaceutically acceptable salt thereof, and/or Compound B or a pharmaceutically acceptable salt or solvate thereof are administered as pro-drugs. Pharmaceutically acceptable pro-drugs of the compounds of the invention are readily prepared by those of skill in the art.

When referring to a dosing protocol, the term “day”, “per day” and the like, refer to a time within one calendar day which begins at midnight and ends at the following midnight.

By the term “treating” and derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine. “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and derivatives thereof, as used herein is meant either, simultaneous administration or any manner of separate sequential administration of a therapeutically effective amount of Compound A, or a pharmaceutically acceptable salt thereof, and Compound B or a pharmaceutically acceptable salt or solvate thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable salt thereof, and Compound B, or a pharmaceutically acceptable salt or solvate thereof, according to the invention. When both compounds are administered simultaneously, the combination kit can contain Compound A, or a pharmaceutically acceptable salt thereof, and Compound B, or a pharmaceutically acceptable salt or solvate thereof, in a single pharmaceutical composition, such as a tablet, or in separate pharmaceutical compositions. When the compounds are not administered simultaneously, the combination kit will contain Compound A, or a pharmaceutically acceptable salt thereof, and Compound B, or a pharmaceutically acceptable salt or solvate thereof, in separate pharmaceutical compositions. The combination kit can comprise Compound A, or a pharmaceutically acceptable salt thereof, and Compound B, or a pharmaceutically acceptable salt or solvate thereof, in separate pharmaceutical compositions in a single package or in separate pharmaceutical compositions in separate packages.

In one aspect there is provided a combination kit comprising the components:

Compound A, or a pharmaceutically acceptable salt thereof, in association with a pharmaceutically acceptable carrier; and

Compound B, or a pharmaceutically acceptable salt or solvate thereof, in association with a pharmaceutically acceptable carrier.

In one embodiment of the invention the combination kit comprises the following components:

Compound A, or a pharmaceutically acceptable salt thereof, in association with a pharmaceutically acceptable carrier; and

Compound B, or a pharmaceutically acceptable salt or solvate thereof, in association with a pharmaceutically acceptable carrier,

wherein the components are provided in a form which is suitable for sequential, separate and/or simultaneous administration.

In one embodiment the combination kit comprises:

a first container comprising Compound A, or a pharmaceutically acceptable salt thereof, in association with a pharmaceutically acceptable carrier; and

a second container comprising Compound B, or a pharmaceutically acceptable salt or solvate thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

As used herein the term “Compound A²” means—Compound A, or a pharmaceutically acceptable salt thereof.

As used herein the term “Compound B²” means—Compound B, or a pharmaceutically acceptable salt or solvate thereof.

The term “loading dose” as used herein will be understood to mean a single dose or short duration regimen of Compound A² or Compound B² having a dosage higher than the maintenance dose administered to the subject to rapidly increase the blood concentration level of the drug. Suitably, a short duration regimen for use herein will be from: 1 to 14 days; suitably from 1 to 7 days; suitably from 1 to 3 days; suitably for three days; suitably for two days; suitably for one day. In some embodiments, the “loading dose” can increase the blood concentration of the drug to a therapeutically effective level. In some embodiments, the “loading dose” can increase the blood concentration of the drug to a therapeutically effective level in conjunction with a maintenance dose of the drug. The “loading dose” can be administered once per day, or more than once per day (e.g., up to 4 times per day). Suitably the “loading dose” will be administered once a day. Suitably, the loading dose will be an amount from 2 to 100 times the maintenance dose; suitably from 2 to 10 times; suitably from 2 to 5 times; suitably 2 times; suitably 3 times; suitably 4 times; suitably 5 times. Suitably, the loading dose will be administered for from 1 to 14 days; suitably from 1 to 7 days; suitably from 1 to 5 days; suitably from 1 to 3 days; suitably for 1 day; suitably for 2 days; suitably for 3 days, followed by a maintenance dosing protocol.

The term “maintenance dose” as used herein will be understood to mean a dose that is serially administered (for example, at least twice), and which is intended to either slowly raise blood concentration levels of the compound to a therapeutically effective level, or to maintain such a therapeutically effective level. The maintenance dose is generally administered once per day and the daily dose of the maintenance dose is lower than the total daily dose of the loading dose.

Suitably the combinations of this invention are administered within a “specified period”.

By the term “specified period” and derivatives thereof, as used herein is meant the interval of time between the administration of one of Compound A² and Compound B² and the other of Compound A² and Compound B². Unless otherwise defined, the specified period can include simultaneous administration. When both compounds of the invention are administered once a day the specified period refers to timing of the administration of Compound A² and Compound B² during a single day. When one or both compounds of the invention are administered more than once a day, the specified period is calculated based on the first administration of each compound on a specific day. All administrations of a compound of the invention that are subsequent to the first during a specific day are not considered when calculating the specific period.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and derivatives thereof, as used herein is meant that both compounds of the invention are administered within a “specified period” for an indicated number of consecutive days, optionally followed by a number of consecutive days where only one of the component compounds is administered.

Regarding “specified period” administration:

Suitably, during the course of treatment, both compounds will be administered within a specified period for at least 1 day—in this case, the duration of time will be at least 1 day; suitably, during the course of treatment, both compounds will be administered within a specified period for at least 2 consecutive days—in this case, the duration of time will be at least 2 days; suitably, during the course of treatment, both compounds will be administered within a specified period for at least 3 consecutive days—in this case, the duration of time will be at least 3 days; suitably, during the course of treatment, both compounds will be administered within a specified period for at least 5 consecutive days—in this case, the duration of time will be at least 5 days; suitably, during the course of treatment, both compounds will be administered within a specified period for at least 7 consecutive days—in this case, the duration of time will be at least 7 days; suitably, during the course of treatment, both compounds will be administered within a specified period for at least 10 consecutive days—in this case, the duration of time will be at least 10 days; suitably, during the course of treatment, both compounds will be administered within a specified period for at least 14 consecutive days—in this case, the duration of time will be at least 14 days; suitably, during the course of treatment, both compounds will be administered within a specified period for at least 21 consecutive days—in this case, the duration of time will be at least 21 days; suitably, during the course of treatment, both compounds will be administered within a specified period for at least 28 consecutive days—in this case, the duration of time will be at least 28 days; suitably, during the course of treatment, both compounds will be administered within a specified period for at least 30 consecutive days—in this case, the duration of time will be at least 30 days. When, during the course of treatment, both compounds are administered within a specified period for over 30 days, the treatment is considered chronic treatment and will continue until an altering event, such as a reassessment in cancer status or a change in the condition of the patient, warrants a modification to the protocol.

Further regarding “specified period” administration:

Suitably, during the course of treatment, both compounds will be administered within a specified period for at least 1 day, followed by the administration of Compound A² alone for at least 1 day—in this case, the duration of time will be at least 2 days; suitably, during the course of treatment, both compounds will be administered within a specified period for at least 1 day, followed by administration of Compound A² alone for at least 2 days—in this case, the duration of time will be at least 3 days; suitably, during the course of treatment, both compounds will be administered within a specified period for at least 1 day, followed by administration of Compound A² alone for at least 3 days—in this case, the duration of time will be at least 4 days; suitably, during the course of treatment, both compounds will be administered within a specified period for at least 1 day, followed by administration of Compound A² alone for at least 4 days—in this case, the duration of time will be at least 5 days; suitably, during the course of treatment, both compounds will be administered within a specified period for at least 1 day, followed by administration of Compound A² alone for at least 5 days—in this case, the duration of time will be at least 6 days; suitably, during the course of treatment, both compounds will be administered within a specified period for at least 1 day, followed by administration of Compound A² alone for at least 6 days—in this case, the duration of time will be at least 7 days; suitably, during the course of treatment, both compounds will be administered within a specified period for at least 1 day, followed by administration of Compound A² alone for at least 7 days—in this case, the duration of time will be at least 8 days; suitably, during the course of treatment, both compounds will be administered within a specified period for at least 2 consecutive days, followed by administration of Compound A² alone for at least 1 day—in this case, the duration of time will be at least 3 days; suitably, during the course of treatment, both compounds will be administered within a specified period for at least 2 consecutive days, followed by administration of Compound A² alone for at least 2 consecutive days—in this case, the duration of time will be at least 4 days; suitably, during the course of treatment, both compounds will be administered within a specified period for at least 2 consecutive days, followed by administration of Compound A² alone for at least 3 consecutive days—in this case, the duration of time will be at least 5 days; suitably, during the course of treatment, both compounds will be administered within a specified period for at least 2 consecutive days, followed by administration of Compound A² alone for at least 4 consecutive days—in this case, the duration of time will be at least 6 days; suitably, during the course of treatment, both compounds will be administered within a specified period for at least 2 consecutive days, followed by administration of Compound A² alone for at least 5 consecutive days—in this case, the duration of time will be at least 7 days; suitably, during the course of treatment, both compounds will be administered within a specified period for at least 2 consecutive days, followed by administration of Compound A² alone for at least 6 consecutive days—in this case, the duration of time will be at least 8 days; suitably, during the course of treatment, both compounds will be administered within a specified period for at least 2 consecutive days, followed by administration of Compound A² alone for at least 7 consecutive days—in this case, the duration of time will be at least 9 days; suitably, during the course of treatment, both compounds will be administered within a specified period for at least 3 consecutive days, followed by administration of Compound A² alone for at least 1 day—in this case, the duration of time will be at least 4 days; suitably, during the course of treatment, both compounds will be administered within a specified period for at least 3 consecutive days, followed by administration of Compound A² alone for at least 2 consecutive days—in this case, the duration of time will be at least 5 days; suitably, during the course of treatment, both compounds will be administered within a specified period for at least 3 consecutive days, followed by administration of Compound A² alone for at least 3 consecutive days—in this case, the duration of time will be at least 6 days; suitably, during the course of treatment, both compounds will be administered within a specified period for at least 3 consecutive days, followed by administration of Compound A² alone for at least 4 consecutive days—in this case, the duration of time will be at least 7 days; suitably, during the course of treatment, both compounds will be administered within a specified period for at least 3 consecutive days, followed by administration of Compound A² alone for at least consecutive days—in this case, the duration of time will be at least 8 days; suitably, during the course of treatment, both compounds will be administered within a specified period for at least 3 consecutive days, followed by administration of Compound A² alone for at least 6 consecutive days—in this case, the duration of time will be at least 9 days; suitably, during the course of treatment, both compounds will be administered within a specified period for at least 3 consecutive days, followed by administration of Compound A² alone for at least 7 consecutive days—in this case, the duration of time will be at least 10 days; suitably, during the course of treatment, both compounds will be administered within a specified period for at least 4 consecutive days, followed by administration of Compound A² alone for at least 1 day—in this case, the duration of time will be at least 5 consecutive days; suitably, during the course of treatment, both compounds will be administered within a specified period for at least 4 consecutive days, followed by administration of Compound A² alone for at least 2 consecutive days—in this case, the duration of time will be at least 6 consecutive days; suitably, during the course of treatment, both compounds will be administered within a specified period for at least 4 consecutive days, followed by administration of Compound A² alone for at least 3 consecutive days—in this case, the duration of time will be at least 7 consecutive days; suitably, during the course of treatment, both compounds will be administered within a specified period for at least 4 consecutive days, followed by administration of Compound A² alone for at least 4 consecutive days—in this case, the duration of time will be at least 8 consecutive days; suitably, during the course of treatment, both compounds will be administered within a specified period for at least 4 consecutive days, followed by administration of Compound A² alone for at least 7 consecutive days—in this case, the duration of time will be at least 11 consecutive days; suitably, during the course of treatment, both compounds will be administered within a specified period for at least 5 consecutive days, followed by administration of Compound A² alone for at least 1 day—in this case, the duration of time will be at least 6 consecutive days; suitably, during the course of treatment, both compounds will be administered within a specified period for at least 5 consecutive days, followed by administration of Compound A² alone for at least 2 consecutive days—in this case, the duration of time will be at least 7 consecutive days; suitably, during the course of treatment, both compounds will be administered within a specified period for at least 5 consecutive days, followed by administration of Compound A² alone for at least 3 consecutive days—in this case, the duration of time will be at least 8 consecutive days; suitably, during the course of treatment, both compounds will be administered within a specified period for at least 5 consecutive days, followed by administration of Compound A² alone for at least 4 consecutive days—in this case, the duration of time will be at least 9 consecutive days; suitably, during the course of treatment, both compounds will be administered within a specified period for at least 5 consecutive days, followed by administration of Compound A² alone for at least 5 consecutive days—in this case, the duration of time will be at least 10 consecutive days; suitably, during the course of treatment, both compounds will be administered within a specified period for at least 7 consecutive days, followed by administration of Compound A² alone for at least 2 consecutive days—in this case, the duration of time will be at least 9 consecutive days; suitably, during the course of treatment, both compounds will be administered within a specified period for at least 14 consecutive days, followed by administration of Compound A² alone for at least 7 consecutive days—in this case, the duration of time will be at least 21 consecutive days; suitably, during the course of treatment, both compounds will be administered within a specified period for at least 30 consecutive days, followed by administration of Compound A² alone for at least 7 consecutive days—in this case, the duration of time will be at least 37 consecutive days. Suitably, during the course of treatment, both compounds will be administered within a specified period for from 1 to 3 consecutive days, followed by administration of Compound A² alone for from 3 to 7 consecutive days. Suitably, during the course of treatment, both compounds will be administered within a specified period for from 3 to 6 consecutive days, followed by administration of Compound A² alone for from 1 to 4 consecutive days. Suitably, during the course of treatment, both compounds will be administered within a specified period for 5 consecutive days, followed by administration of Compound A² alone for 2 consecutive days. Suitably, during the course of treatment, both compounds will be administered within a specified period for 2 consecutive days, followed by administration of Compound A² alone for from 3 to 7 consecutive days. Suitably, during the course of treatment, both compounds will be administered within a specified period for from 1 to 3 days over a 7 day period, and during the other days of the 7 day period Compound A² will be administered alone. Suitably, during the course of treatment, both compounds will be administered within a specified period for 2 days over a 7 day period, and during the other days of the 7 day period Compound A² will be administered alone.

Further regarding “specified period” administration:

Suitably, during the course of treatment, both compounds will be administered within a specified period for at least 1 day, followed by the administration of Compound B² alone for at least 1 day—in this case, the duration of time will be at least 2 days; suitably, during the course of treatment, both compounds will be administered within a specified period for at least 1 day, followed by administration of Compound B² alone for at least 2 days—in this case, the duration of time will be at least 3 days; suitably, during the course of treatment, both compounds will be administered within a specified period for at least 1 day, followed by administration of Compound B² alone for at least 3 days—in this case, the duration of time will be at least 4 days; suitably, during the course of treatment, both compounds will be administered within a specified period for at least 1 day, followed by administration of Compound B² alone for at least 4 days—in this case, the duration of time will be at least 5 days; suitably, during the course of treatment, both compounds will be administered within a specified period for at least 1 day, followed by administration of Compound B² alone for at least 5 days—in this case, the duration of time will be at least 6 days; suitably, during the course of treatment, both compounds will be administered within a specified period for at least 1 day, followed by administration of Compound B² alone for at least 6 days—in this case, the duration of time will be at least 7 days; suitably, during the course of treatment, both compounds will be administered within a specified period for at least 1 day, followed by administration of Compound B² alone for at least 7 days—in this case, the duration of time will be at least 8 days; suitably, during the course of treatment, both compounds will be administered within a specified period for at least 2 consecutive days, followed by administration of Compound B² alone for at least 1 day—in this case, the duration of time will be at least 3 days; suitably, during the course of treatment, both compounds will be administered within a specified period for at least 2 consecutive days, followed by administration of Compound B² alone for at least 2 consecutive days—in this case, the duration of time will be at least 4 days; suitably, during the course of treatment, both compounds will be administered within a specified period for at least 2 consecutive days, followed by administration of Compound B² alone for at least 3 consecutive days—in this case, the duration of time will be at least 5 days: suitably, during the course of treatment, both compounds will be administered within a specified period for at least 2 consecutive days, followed by administration of Compound B² alone for at least 4 consecutive days—in this case, the duration of time will be at least 6 days; suitably, during the course of treatment, both compounds will be administered within a specified period for at least 2 consecutive days, followed by administration of Compound B² alone for at least 5 consecutive days—in this case, the duration of time will be at least 7 days; suitably, during the course of treatment, both compounds will be administered within a specified period for at least 2 consecutive days, followed by administration of Compound B² alone for at least 6 consecutive days—in this case, the duration of time will be at least 8 days; suitably, during the course of treatment, both compounds will be administered within a specified period for at least 2 consecutive days, followed by administration of Compound B² alone for at least 7 consecutive days—in this case, the duration of time will be at least 9 days; suitably, during the course of treatment, both compounds will be administered within a specified period for at least 3 consecutive days, followed by administration of Compound B² alone for at least 1 day—in this case, the duration of time will be at least 4 days; suitably, during the course of treatment, both compounds will be administered within a specified period for at least 3 consecutive days, followed by administration of Compound B² alone for at least 2 consecutive days—in this case, the duration of time will be at least 5 days; suitably, during the course of treatment, both compounds will be administered within a specified period for at least 3 consecutive days, followed by administration of Compound B² alone for at least 3 consecutive days—in this case, the duration of time will be at least 6 days; suitably, during the course of treatment, both compounds will be administered within a specified period for at least 3 consecutive days, followed by administration of Compound B² alone for at least 4 consecutive days—in this case, the duration of time will be at least 7 days; suitably, during the course of treatment, both compounds will be administered within a specified period for at least 3 consecutive days, followed by administration of Compound B² alone for at least 5 consecutive days—in this case, the duration of time will be at least 8 days; suitably, during the course of treatment, both compounds will be administered within a specified period for at least 3 consecutive days, followed by administration of Compound B² alone for at least 6 consecutive days—in this case, the duration of time will be at least 9 days; suitably, during the course of treatment, both compounds will be administered within a specified period for at least 3 consecutive days, followed by administration of Compound B² alone for at least 7 consecutive days—in this case, the duration of time will be at least 10 days; suitably, during the course of treatment, both compounds will be administered within a specified period for at least 4 consecutive days, followed by administration of Compound B² alone for at least 1 day—in this case, the duration of time will be at least 5 consecutive days; suitably, during the course of treatment, both compounds will be administered within a specified period for at least 4 consecutive days, followed by administration of Compound B² alone for at least 2 consecutive days—in this case, the duration of time will be at least 6 consecutive days; suitably, during the course of treatment, both compounds will be administered within a specified period for at least 4 consecutive days, followed by administration of Compound B² alone for at least 3 consecutive days—in this case, the duration of time will be at least 7 consecutive days; suitably, during the course of treatment, both compounds will be administered within a specified period for at least 4 consecutive days, followed by administration of Compound B² alone for at least 4 consecutive days—in this case, the duration of time will be at least 8 consecutive days; suitably, during the course of treatment, both compounds will be administered within a specified period for at least 4 consecutive days, followed by administration of Compound B² alone for at least 7 consecutive days—in this case, the duration of time will be at least 11 consecutive days; suitably, during the course of treatment, both compounds will be administered within a specified period for at least 5 consecutive days, followed by administration of Compound B² alone for at least 1 day—in this case, the duration of time will be at least 6 consecutive days; suitably, during the course of treatment, both compounds will be administered within a specified period for at least 5 consecutive days, followed by administration of Compound B² alone for at least 2 consecutive days—in this case, the duration of time will be at least 7 consecutive days; suitably, during the course of treatment, both compounds will be administered within a specified period for at least 5 consecutive days, followed by administration of Compound B² alone for at least 3 consecutive days—in this case, the duration of time will be at least 8 consecutive days; suitably, during the course of treatment, both compounds will be administered within a specified period for at least 5 consecutive days, followed by administration of Compound B² alone for at least 4 consecutive days—in this case, the duration of time will be at least 9 consecutive days; suitably, during the course of treatment, both compounds will be administered within a specified period for at least 5 consecutive days, followed by administration of Compound B² alone for at least 5 consecutive days—in this case, the duration of time will be at least 10 consecutive days; suitably, during the course of treatment, both compounds will be administered within a specified period for at least 7 consecutive days, followed by administration of Compound B² alone for at least 2 consecutive days—in this case, the duration of time will be at least 9 consecutive days; suitably, during the course of treatment, both compounds will be administered within a specified period for at least 14 consecutive days, followed by administration of Compound B² alone for at least 7 consecutive days—in this case, the duration of time will be at least 21 consecutive days; suitably, during the course of treatment, both compounds will be administered within a specified period for at least 30 consecutive days, followed by administration of Compound B² alone for at least 7 consecutive days—in this case, the duration of time will be at least 37 consecutive days. Suitably, during the course of treatment, both compounds will be administered within a specified period for from 1 to 3 consecutive days, followed by administration of Compound B² alone for from 3 to 7 consecutive days. Suitably, during the course of treatment, both compounds will be administered within a specified period for from 3 to 6 consecutive days, followed by administration of Compound B² alone for from 1 to 4 consecutive days. Suitably, during the course of treatment, both compounds will be administered within a specified period for 5 consecutive days, followed by administration of Compound B² alone for 2 consecutive days. Suitably, during the course of treatment, both compounds will be administered within a specified period for 2 consecutive days, followed by administration of Compound B² alone for from 3 to 7 consecutive days. Suitably, during the course of treatment, both compounds will be administered within a specified period for from 1 to 3 days over a 7 day period, and during the other days of the 7 day period Compound B² will be administered alone. Suitably, during the course of treatment, both compounds will be administered within a specified period for 2 days over a 7 day period, and during the other days of the 7 day period Compound B² will be administered alone.

Further regarding “specified period” administration:

Suitably, during the course of treatment, Compound A² and Compound B² will be administered within a specified period for from 1 to 3 days over a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for from 1 to 3 days over a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 3 days over a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 3 days over a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 2 days over a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 2 days over a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 1 day during a 7 day period, and during the other days of the 7 day period Compound A² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for 1 day during a 7 day period, and during the other days of the 7 day period Compound B² will be administered alone. Suitably, this 7 day protocol is repeated for 2 cycles or for 14 days; suitably for 4 cycles or 28 days; suitably for continuous administration.

Suitably, during the course of treatment, Compound A² and Compound B² will be administered within a specified period for from 1 to 5 days over a 14 day period, and during the other days of the 14 day period Compound A² will be administered alone. Suitably, this 14 day protocol is repeated for 2 cycles or for 28 days; suitably for continuous administration.

Suitably, during the course of treatment, Compound A² and Compound B² will be administered within a specified period for from 1 to 5 days over a 14 day period, and during the other days of the 14 day period Compound B² will be administered alone. Suitably, this 14 day protocol is repeated for 2 cycles or for 28 days; suitably for continuous administration.

Suitably, if the compounds are not administered during a “specified period”, they are administered sequentially. By the term “sequential administration”, and derivates thereof, as used herein is meant that one of Compound A² and Compound B² is administered for 1 or more consecutive days and the other of Compound A² and Compound B² is subsequently administered for 1 or more consecutive days. Unless otherwise defined, the “sequential administration” and in all dosing protocols described herein, do not have to commence with the start of treatment and terminate with the end of treatment, it is only required that the administration of one of Compound A² and Compound B² followed by the administration of the other of Compound A² and Compound B², or the indicated dosing protocol, occur at some point during the course of treatment. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding Sequential Administration:

Suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 2 to 7 consecutive days, followed by a drug holiday of from 2 to 10 days, followed by administration of the other of Compound A² and Compound B² for from 2 to 7 consecutive days.

Suitably, Compound B² will be administered first in the sequence, followed by an optional drug holiday, followed by administration of Compound A². Suitably, Compound B² is administered for from 1 to 21 consecutive days, followed by an optional drug holiday, followed by administration of Compound A² for from 1 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

Suitably, Compound A² will be administered first in the sequence, followed by an optional drug holiday, followed by administration of Compound B². Suitably, Compound A² is administered for from 1 to 21 consecutive days, followed by an optional drug holiday, followed by administration of Compound B² for from 1 to 21 consecutive days. Suitably, Compound A² is administered for from 3 to 21 consecutive days, followed by a drug holiday of from 1 to 14 days, followed by administration of Compound B² for from 3 to 21 consecutive days. Suitably, Compound A² is administered for from 3 to 21 consecutive days, followed by a drug holiday of from 3 to 14 days, followed by administration of Compound B² for from 3 to 21 consecutive days. Suitably, Compound A² is administered for 21 consecutive days, followed by an optional drug holiday, followed by administration of Compound B² for 14 consecutive days. Suitably, Compound A² is administered for 14 consecutive days, followed by a drug holiday of from 1 to 14 days, followed by administration of Compound B² for 14 consecutive days. Suitably, Compound A² is administered for 7 consecutive days, followed by a drug holiday of from 3 to 10 days, followed by administration of Compound B² for 7 consecutive days. Suitably, Compound A² is administered for 3 consecutive days, followed by a drug holiday of from 3 to 14 days, followed by administration of Compound B² for 7 consecutive days. Suitably, Compound A² is administered for 3 consecutive days, followed by a drug holiday of from 3 to 10 days, followed by administration of Compound B² for 3 consecutive days. Suitably, Compound A² is administered for 7 consecutive days, followed by administration of Compound B² for 1 day. Suitably, Compound A² is administered for 6 consecutive days, followed by administration of Compound B² for 1 day. Suitably, Compound B² is administered for 1 day, followed by administration of Compound A² for 7 consecutive days. Suitably, Compound B² is administered for 1 day, followed by administration of Compound A² for 6 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 10 mg to about 1,000 mg; suitably, the amount will be selected from about 20 mg to about 900 mg; suitably, the amount will be selected from about 20 mg to about 800 mg; suitably, the amount will be selected from about 20 mg to about 500 mg; suitably, the amount will be 20 mg; suitably. the amount will be 40 mg; suitably, the amount will be 60 mg; suitably, the amount will be 80 mg; suitably, the amount will be 100 mg; suitably, the amount will be 120 mg; suitably, the amount will be 140 mg; suitably, the amount will be 160 mg; suitably, the amount will be 180 mg; suitably, the amount will be 200 mg; suitably, the amount will be 220 mg; suitably, the amount will be 250 mg; suitably, the amount will be 270 mg; suitably, the amount will be 290 mg; suitably, the amount will be 310 mg; suitably, the amount will be 330 mg; suitably, the amount will be 350 mg; suitably, the amount will be 370 mg; suitably, the amount will be 390 mg; suitably, the amount will be 410 mg; suitably, the amount will be 450 mg; suitably, the amount will be 500 mg; suitably, the amount will be 550 mg; suitably, the amount will be 600 mg; suitably, the amount will be 650 mg; suitably, the amount will be 700 mg; suitably, the amount will be 750 mg; suitably, the amount will be 800 mg; suitably, the amount will be 850 mg; suitably, the amount will be 900 mg; suitably, the amount will be 950 mg; suitably, the amount will be 1000 mg. Suitably, the selected amount of Compound A² is administered from 1 to 4 times a day. Suitably, the selected amount of Compound A² is administered twice a day. Suitably, the selected amount of Compound A² is administered once a day.

Suitably, the amount of Compound B² administered as part of the combination according to the present invention will be an amount selected from about 0.1 mg to about 5 mg; suitably, the amount will be selected from about 0.125 mg to about 4 mg; suitably, the amount will be selected from about 0.125 mg to about 3 mg; suitably, the amount will be selected from about 0.125 mg to about 2 mg. For example, the amount of Compound B² administered as part of the combination according to the present invention can be 0.1 mg, 0.125 mg, 0.25 mg, 0.375 mg, 0.5 mg, 0.625 mg, 0.75 mg, 0.875 mg, 1.0 mg, 1.125 mg, 1.25 mg, 1.5 mg, 1.75 mg, 2.0 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg. Suitably, the selected amount of Compound B² is administered from 1 to 3 times a day, in one or more tablets. Suitably, the selected amount of Compound B² is administered twice a day, in one or more tablets. Suitably, the selected amount of Compound B² is administered once a day, in one or more tablets.

As used herein, all amounts specified for Compound A² and Compound B² are indicated as the administered amount of free or unsalted compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation. Suitably, Compound A² and Compound B² are administered in separate pharmaceutical compositions.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, suitably, may be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

The combinations of the invention are tested for efficacy, advantageous and synergistic properties generally according to known procedures.

Suitably, the present invention relates to a method for treating or lessening the severity of a cancer selected from: brain (gliomas), glioblastomas, astrocytomas, glioblastoma multiforme, Bannayan-Zonana syndrome. Cowden disease. Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, mesothelioma may include malignant advanced mesothelioma, malignant NOS mesothelioma, malignant pleural mesothelioma, mesothelioma with MAPK pathway activation, recurrent or progressive, and/or unresectable malignant pleural mesothelioma with measurable lesion.

Suitably, the present invention relates to a method for treating or lessening the severity of a cancer selected from: Mesothelioma, Lung, Melanoma, Glioblastoma, Thyroid, Breast, Pancreatic, Renal cell carcinoma, Ovarian, Head and Neck, Endometrial.

Suitably, the present invention relates to a method for treating or lessening the severity of a cancer selected from ovarian, breast, lung, mesothelioma, and glioblastomas.

This invention provides a combination comprising 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof.

This invention also provides for a combination comprising 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, for use in therapy.

This invention also provides for a combination comprising 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, for use in treating cancer.

This invention also provides a pharmaceutical composition comprising a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof.

This invention also provides a combination kit comprising 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof.

This invention also provides for the use of a combination comprising 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, in the manufacture of a medicament.

This invention also provides for the use of a combination comprising 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, in the manufacture of a medicament to treat cancer.

This invention also provides a method of treating cancer which comprises administering a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, suitably the dimethyl sulfoxide solvate thereof, to a subject in need thereof.

### EXPERIMENTAL

### Reagents and Methods

Human Cell Lines

Five human mesothelioma cell lines were used in these studies. Cells were grown in RPMI1640 media containing 10% FBS, 1% L-glutamine, 1% sodium pyruvate under standard cell culture conditions. The mesothelioma cell lines: Mero-14, Mero-82, NCI-H2052, NO36, and ONE58.

Anchorage-Independent Growth-Death Assay

The cellular response to Compound A or B was evaluated in an anchorage-independent cell growth assay that quantified the extent of cell growth inhibition and the net change in cell population. The assay was performed in black, clear bottom, untreated 384-well plates (Greiner #781096). It is important to use either non-tissue culture treated or Low Attachment plates to prevent cells from adhering to the plate during the assay. In brief, the assay was performed as described below.

A 1% (weight/volume) stock of methylcellulose solution was prepared by dissolving 5 grams of methylcellulose (Sigma #M0512) in 495 mL of cell culture medium. Here, RPMI1640 media containing 10% FBS, 1% L-glutamine, 1% sodium pyruvate was added to the cooled methylcellulose that had been placed in a glass container and autoclaved to sterilize. Media can be substituted if the cells require different cell culture medium for growth. The dissolution often took a day with vigorous stirring at 4° C. maintaining sterile conditions.

Cells were plated into a 384 well plate with assay conditions of 0.65% methylcellulose (final concentration) and 1000 cells per well in a final volume of 48 μL. This was achieved by diluting cells harvested from culture and re-suspended in growth medium (dilute to 2.0833×10⁴ cells/mL) with the 1% methylcellulose. The cells were mixed by inversion to distribute evenly, bubbles were dispersed and 48 μL was placed into the well with a positive displacement pipette. The plates were placed in a cell culture incubator containing 5% CO₂ at 37° C.

Serial dilution of compound in DMSO was done in a 384 well plate starting with μL of stock compound solution in the first column and 10 μL of DMSO in the other wells. Ten μL from the compound well was transferred into the DMSO containing well, mixed, and the serial dilution was continued with 10 μL transfers across the plate. Then, 4 μL of this DMSO diluted compound was added into wells of a new 384 well plate containing 105 μL of appropriate growth medium. This ‘compound plate’ was used to dose the assay plates containing cells in methylcellulose.

For combination experiments, 20 μL of stock compound solution was a mixture of Compound A and Compound B at the designated ratio. For an example, for 1 to 1 ratio, 10 ul of Compound A at 40 mM and 10 ul of Compound B at 40 mM was mixed to generate ul of stock compound solution with both Compound A and Compound B at 20 mM concentration. For an 8 to 1 ratio, 10 ul of Compound A at 40 mM, 5 ul of Compound B at 10 mM plus 5 ul of DMSO were mixed for a 20 ul stock compound solution with the Compound A concentration at 20 mM and Compound B at 2.5 mM. Serial dilution and compound plate dilution was done as described above.

Two μL from each well of the ‘compound plate’ was added to the individual wells of the ‘cell plates’ containing the 48 μL of cells in methylcellulose to initiate the assay. These assay plates were placed in the cell culture incubator for 6 days. One ‘cell plate’ was selected at random and was developed (see below) with Cell TiterGlo (CTG) at the time compound was added to the remaining cell plates and represents the time equal zero (T0) plate, i.e. it represents the number of cells at the time of compound addition.

After 6 days the assay was stopped and the plates were developed by placing a black sticker on bottom of each plate to block light, 25 μL of CTG was added and then plates were incubate for 20 minutes at room temperature. The plates were scanned using a luminescence protocol on the EnVision (Perkin-Elmer).

Results were expressed as a percent of the T0 value and plotted against the compound concentration. All values had a ‘no cell’ background subtraction and the T0 value was normalized to 100% and represents the number of cells at the time of compound addition. The cellular response was determined by fitting the concentration response curves using a 4-parameter curve fit equation and determining the concentration that inhibited growth by 50% (gIC₅₀). The gIC₅₀ value is the midpoint of the growth window (between T0 and growth of DMSO controls). The gIC₁₀₀ value is the concentration of compound required for 100% growth inhibition. The measure of net change in the population was quantified by the Ymin-T0 value that was determined by subtracting the T0 value (100%) from the Ymin value (%) that was determined from the fit of the concentration response curve. Positive values indicate net cell growth and negative values represent net cell kill. In order to quantify compound concentration required to induce cell death, the death EC₅₀ (dEC₅₀) was determined and defined as the concentration of compound that caused a 50% reduction in the cell population relative to the T0 value.

The results for the combination were analyzed by determination of Combination Index (CI) values using both mutually exclusive and mutually non-exclusive assumptions (12).

The mutually exclusive equation:

$$CI = \frac{{XC}_{50} of A + B}{{XC}_{50} of A} + \frac{{XC}_{50} of B + A}{{XC}_{50} of B}$$

and mutually non-exclusive:

$$CI = \frac{{XC}_{50} of A + B}{{XC}_{50} of A} + \frac{{XC}_{50} of B + A}{{XC}_{50} of B} + \frac{( {XC}_{50} of A + B ) ( {XC}_{50} of B + A )}{( {XC}_{50} of A ) ( {XC}_{50} of B )}$$

The XC₅₀ denotes that each parameter from the growth-death analysis was used, either gIC₅₀ or gIC₁₀₀ or dEC₅₀ values, to calculate the CI values. The A+B represents the XC₅₀ value for the combination relative to the concentration of A and the B+A represents the XC₅₀ value for the combination relative to the concentration of B in the combination.

Results

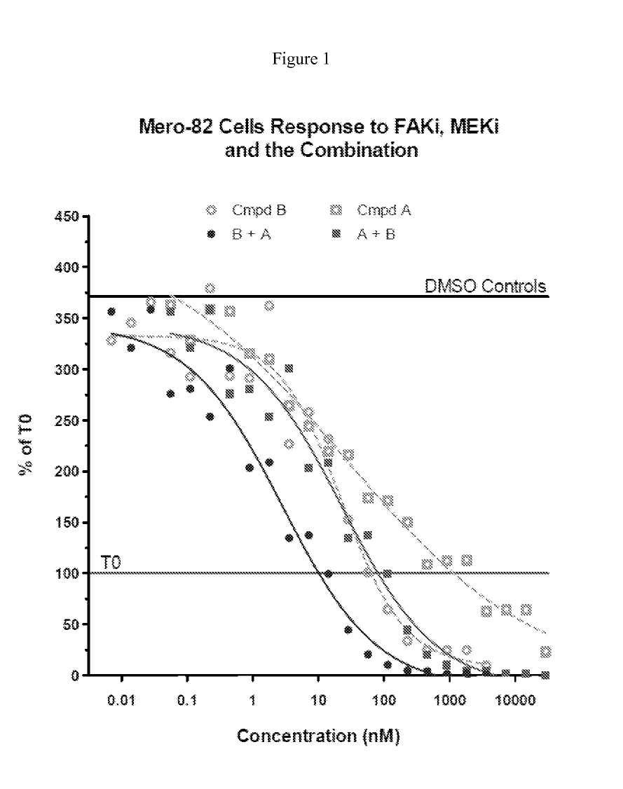

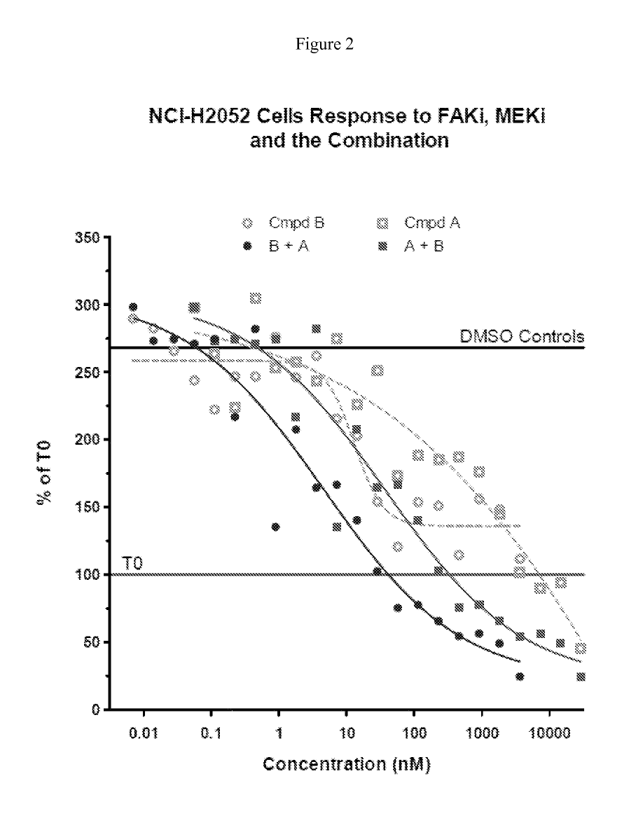

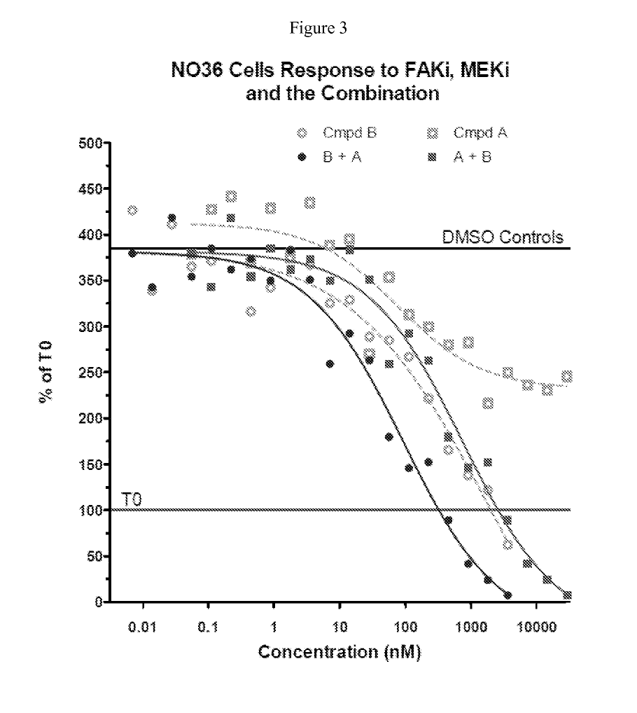

The FAK inhibitor (FAKi, Compound A) and the MEK inhibitor (MEKi, Compound B) and combination of the FAKi and MEKi were evaluated in five human mesothelioma cell lines in the anchorage-independent growth-death assay. Concentration-response curves for three of the cell lines are shown in FIGS. 1 to 3. FIG. 1 has the response for the Mero-82 cell line for each single agent and the combination. The concentration-response curve for the combination is illustrated twice, representing the response in relation to the concentration of either Compound A in the combination or the concentration of Compound B in the combination. FIG. 2 represents the response for the NCI-H2052 mesothelioma cells and FIG. 3 the NO36 cells. For all three cell lines, the curve fits for the combination was shifted to lower concentrations (‘left shifted’) relative to the respective single agent activity. These results indicate a clear benefit for the combination relative to the single agent treatments.

Parameters derived from the curve fits to quantify the cellular growth and death responses are shown in Table 1 for all five mesothelioma cell lines. Table 1A illustrates the Compound A single agent parameters and Table 1B has the results for the combination of Compound A with Compound B. The values for the derived parameters in Table 1B are based on concentrations of Compound A in the combination. The single agent activity for Compound B is shown in Table 1C and Table 1D has the results for the combination of Compound B with Compound A. The values in Table 1 D are concentrations of Compound B in the combination. From these values, the Combination Index (CI) values were determined and shown in Table 2.

The CI values indicate the combination of the FAK inhibitor and MEK inhibitor resulted in synergistic growth inhibition in 4 of the 5 mesothelioma cell lines grown in anchorage-independent conditions. The smaller the CI value the more synergistic the combination. The conclusion of potent synergy was the same regardless of the exclusivity assumption as can be seen by comparing the data from the mutually exclusive calculation (Table 2A) and the mutually non-exclusive calculation (Table 2B). The synergistic response was observed for growth inhibition with CI values for both the gIC₅₀ and gIC₁₀₀ values, consistent with the ‘left-shifted’ concentration-response curves (FIG. 1).

In order to relate the synergistic responses defined by the CI values to biological response and compound concentration, the difference in compound concentration was determined between the combination and each single agent to achieve an equivalent biological response (Table 3). Table 3A shows the fold reduction of Compound A when used in combination with Compound B compared to Compound A used alone for growth inhibition (gIC₅₀ and gIC₁₀₀ values) and for the induction of cell death (dEC₅₀ value). Table 3B illustrates the fold reduction of Compound B used in combination with Compound A compared to Compound B used alone. For the cell lines and parameters evaluated, there was on average a ˜30 fold reduction for the FAK inhibitor and ˜5 fold reduction for the MEK inhibitor when used in combination compared to single agents for achieving the equivalent response of growth inhibition and cell death.

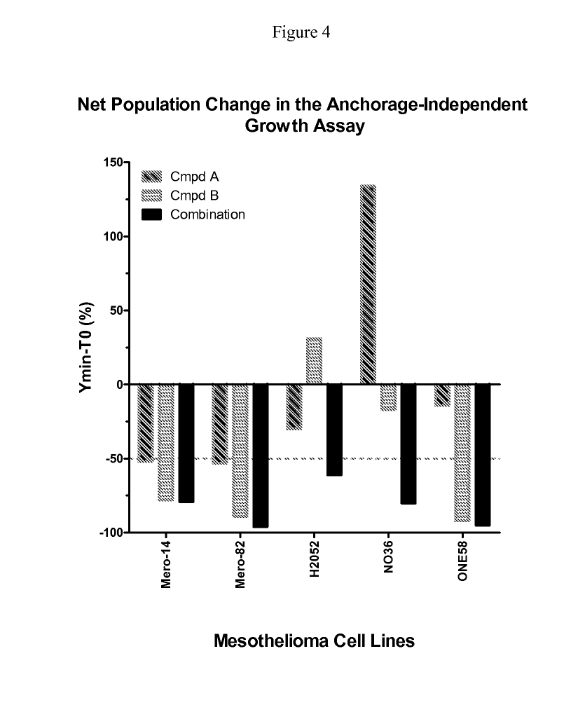

The anchorage-independent growth-death assay had 6 days of compound exposure on the cells. The net change in cell population during this treatment was quantified with the Ymin-T0 parameter. FIG. 4 plots the Ymin-T0 value for each cell line with Compound A, Compound B, and the combination. Compound A was generally cytostatic although the Mero-14 and Mero-82 cell lines had evidence of net cell kill (Ymin-T0 of <−50%). Compound B proved to be cytotoxic in three of the five cell lines (Ymin-T0<−50%) but the combination of Compound A and Compound B resulted in net cell kill for all five mesothelioma cell lines (FIG. 4).

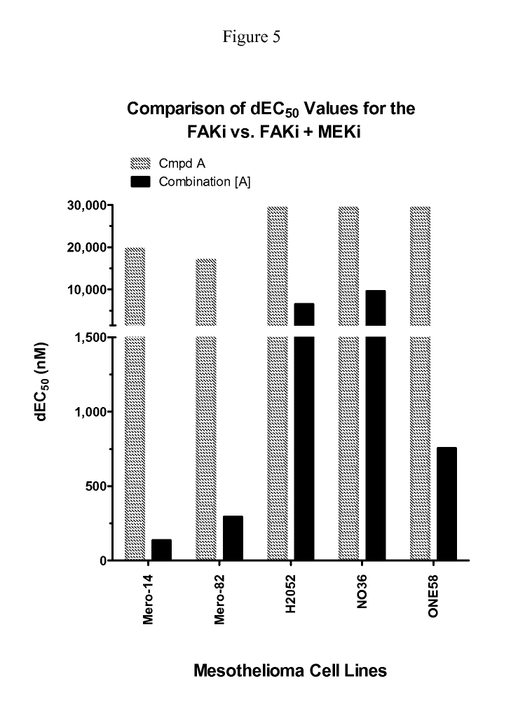

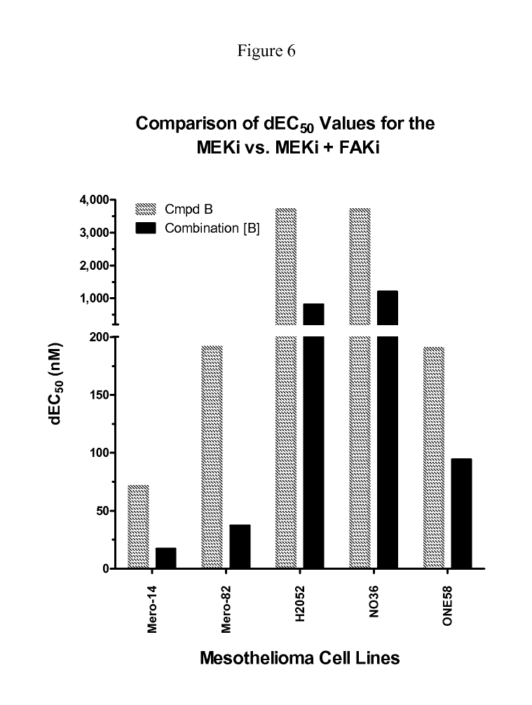

The enhanced cell death observed with the Compound A and Compound B combination was also quantified in relation to the amount of compound required to induce 50% net cell kill (dEC₅₀) and directly compared to each single agent (FIGS. 5 and 6). FIG. 5 plots the dEC₅₀ for Compound A compared with the dEC₅₀ for the Compound A and Compound B combination and FIG. 6 plots the dEC₅₀ for Compound B compared with the dEC₅₀ for the Compound B and Compound A combination. The amount of either agent in the combination was greatly reduced compared to either single agent alone, consistent with the observed synergistic activity in the mesothelioma cell lines.

### Tables

TABLE 1
Parameters determined from the concentration-response curves for the
mesothelioma cell lines analyzed in the anchorage-independent growth-death assay. Table
1A has the parameter values from Compound A used as a single agent and Table 1B the
values from the combination of Compound A and Compound B with respect to the
concentration of Compound A in the mixture. Table 1C has the parameter values from
Compound B used as a single agent and Table 1D the values from the combination of
Compound B and Compound A with respect to the concentration of Compound B in the
mixture.
A
Compound A single agent

| | | | | | Ymin- | Cell | High |
|---|---|---|---|---|---|---|---|
| | | | gIC₁₀₀ | dEC₅₀ | T0 | Population | Conc. |
| | Cell Line | gIC₅₀ (nM) | (nM) | (nM) | (%) | Doubling | (nM) |
| Compound A | MERO-14 | 56 | 3888 | 9581 | −52 | 1.87 | 29326 |
| Compound A | MERO-82 | 16 | 983 | 6900 | −53 | 1.86 | 29326 |
| Compound A | NCI-H2052 | 408 | 5730 | | −30 | 1.45 | 29326 |
| Compound A | NO36 | 17047 | | | 134 | 1.93 | 29326 |
| Compound A | ONE58 | 23 | 6135 | | −14 | 1.16 | 29326 |

B

| | Values determined | | | | | |
|---|---|---|---|---|---|---|
| | from concentration | | | | | |
| | of cmpd A | | | | | |
| | in the combination | | | | Cell | High |
| | gIC₅₀ | gIC₁₀₀ | dEC₅₀ | Ymin-T0 | Population | Conc. |
| Cell Line | (nM) | (nM) | (nM) | (%) | Doubling | (nM) |
| MERO-14 | 7 | 41 | 136 | −79 | 1.90 | 29326 |
| MERO-82 | 6 | 73 | 293 | −96 | 1.89 | 29326 |
| NCI-H2052 | 17 | 300 | 6447 | −61 | 1.44 | 29326 |
| NO36 | 214 | 2625 | 9538 | −80 | 1.94 | 29326 |
| ONE58 | 39 | 188 | 755 | −95 | 1.16 | 29326 |

C
Compound B single
agent

| | | | | | Ymin- | Cell | |
|---|---|---|---|---|---|---|---|
| | Cell | gIC₅₀ | gIC₁₀₀ | dEC₅₀ | T0 | Population | High Conc. |
| | Line | (nM) | (nM) | (nM) | (%) | Doubling | (nM) |
| Compound B | MERO- | 6.6 | 26 | 71 | −78 | 1.93 | 3666 |
| | 14 | | | | | | |
| Compound B | MERO- | 10 | 67 | 191 | −89 | 1.89 | 3666 |
| | 82 | | | | | | |
| Compound B | NCI-H2052 | 16 | | | 31 | 1.42 | 3666 |
| Compound B | NO36 | 134 | 2345 | | −17 | 1.94 | 3666 |
| Compound B | ONE58 | 8.8 | 45 | 190 | −92 | 1.16 | 3666 |

D

| | Values | | | | | |
|---|---|---|---|---|---|---|
| | determined from the | | | | | |
| | concentration of Cmpd | | | | | |
| | B in the combination | | | | Cell | |
| | gIC₅₀ | gIC₁₀₀ | dEC₅₀ | Ymin-T0 | Population | High |
| Cell Line | (nM) | (nM) | (nM) | (%) | Doubling | Conc. (nM) |
| MERO-14 | 0.91 | 5 | 17 | −79 | 1.90 | 3666 |
| MERO-82 | 0.70 | 9 | 37 | −96 | 1.89 | 3666 |
| NCI-H2052 | 2.1 | 37 | 806 | −61 | 1.44 | 3666 |
| NO36 | 27 | 328 | 1192 | −80 | 1.94 | 3666 |
| ONE58 | 5 | 23 | 94 | −95 | 1.16 | 3666 |

TABLE 2
The combination index (CI) values were calculated for each of the parameters
(gIC₅₀, gIC₁₀₀, dEC₅₀) determined from the concentration-response curves for each cell
line. A synergistic response is reflected in values of <0.85, an additive response for values
of 0.85-1.2, and an antagonistic response with values >1.2. CI values could not he
determined if one of the four parameters could not be derived from the
concentration-response curve. A) CI values determined assuming a mutually exclusive
interaction of the compounds and B) CI values determined assuming a mutually
non-exclusive interaction.
A

| | mutually exclusive 0 values | | |
|---|---|---|---|
| | CI for | CI for | CI for |
| Cell Line | gIC₅₀ | gIC₁₀₀ | dEC₅₀ |
| MERO-14 | 0.27 | 0.21 | 0.25 |
| MERO-82 | 0.42 | 0.21 | 0.21 |
| NCI-H2052 | 0.17 | | |
| NO36 | 0.21 | | |
| ONE58 | 2.27 | 0.55 | |

B

| | mutually non-exclusive CI values | | |
|---|---|---|---|
| | CI for | CI for | CI for |
| Cell Line | gIC₅₀ | gIC₁₀₀ | dEC₅₀ |
| MERO-14 | 0.29 | 0.21 | 0.25 |
| MERO-82 | 0.45 | 0.22 | 0.21 |
| NCI-H2052 | 0.17 | | |
| NO36 | 0.21 | | |
| ONE58 | 3.23 | 0.56 | |

TABLE 3
Calculation of the fold change in compound concentration for the
combination compared to each single agent for the same
biological response of growth inhibition (gIC₅₀
and gIC₁₀₀) and net cell kill (dEC₅₀). A) The fold
reduction of Compound A in combination relative to Compound
A as a single agent and B) the fold reduction of Compound B
in combination relative to Compound B as a single agent.
A

| | Fold reduction of FAKI | | |
|---|---|---|---|
| | gIC₅₀ | gIC₁₀₀ | dEC₅₀ |
| Cell Line | shift | shift | shift |
| MERO-14 | 7.7 | 96 | 144 |
| MERO-82 | 2.8 | 14 | 58 |
| NCI-H2052 | 25 | 19 | |
| NO36 | 80 | | |
| ONE58 | 0.6 | 33 | |

B

| | Fold reduction of MEKI | | |
|---|---|---|---|
| | gIC₅₀ | gIC₁₀₀ | dEC₅₀ |
| Cell Line | shift | shift | shift |
| MERO-14 | 7.3 | 5.2 | 4.2 |
| MERO-82 | 14 | 7.4 | 5.2 |
| NCI-H2052 | 7.6 | | |
| NO36 | 5.0 | 7.1 | |
| ONE58 | 1.8 | 2.0 | 2.0 |

### FIGURES

FIGS. 1 to 3

Concentration-response curves for Compound A ( ), Compound B (∘), the combination of Compound A+Compound B with respect to Compound A concentration (▪), and the combination with respect to Compound B concentration (•) in FIG. 1) Mero-82 cells, FIG. 2) NCI-H2052 cells, and FIG. 3) NO36 cells. The molar ratio was 8:1 (Compound A:Compound B) in this fixed ratio concentration-response analysis. The horizontal line at 100% and labeled T0 represents the number of cells at the time of compound addition and the horizontal lines between ˜275% and ˜375% represents the growth of control cells treated only with DMSO.

FIG. 4

The net change in the cell population for Compound A ( [symbol] ) and Compound B ( [symbol] ) as single agents or in combination ( [symbol] ). A Ymin-T0 value of 0% indicates no net change in cell number for the duration of the assay, negative values indicate net cell kill and positive values indicate an increase in cell number during the experiment.

FIGS. 5 and 6

The death EC₅₀ (dEC₅₀) values for each single agent and the FAK inhibitor and MEK inhibitor combination in mesothelioma cell lines. FIG. 5) Compound A dEC₅₀ values ( [symbol] ) compare with the dEC₅₀ values from the Compound A and Compound B combination ( [symbol] ) and FIG. 6) the Compound B dEC₅₀ values ( [symbol] ) compared with the dEC₅₀ values from the Compound B and Compound A combination ( [symbol] ). In cases, where a dEC₅₀ value could not be determined because of the lack of activity, the highest compound concentration used in the assay was used to visualize on the graph.

1. A combination comprising:
(i) a compound of Structure (I): 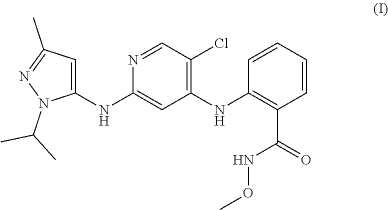 [SMILES: CONC(=O)c1ccccc1Nc1cc(Nc2cc(C)nn2C(C)C)ncc1Cl]
or a pharmaceutically acceptable salt thereof; and
(ii) a compound of Structure (II): 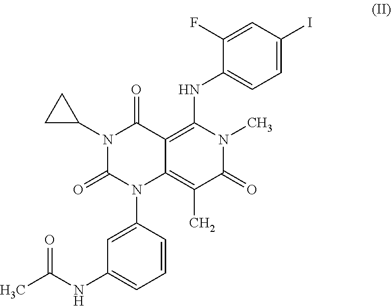 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

2. A combination kit comprising a combination according to claim 1 together with a pharmaceutically acceptable carrier or carriers.

3. A combination according to claim 1 where the amount of the compound of Structure (I) is an amount selected from 10 mg to 1,000 mg, and that amount is administered once or twice per day, and the amount of the compound of Structure (II) is an amount selected from 0.1 mg to 5 mg, and that amount is administered once per day.

4. (canceled)

5. A method of treating cancer in a human in need thereof which comprises the in vivo administration of a therapeutically effective amount of a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, to such human, wherein the combination is administered within a specified period, and wherein the combination is administered for a duration of time.

6. A method according to claim 5 wherein the amount of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, is selected from about 20 mg to about 800 mg, and that amount is administered once or twice per day, and the amount of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt of solvate thereof, is selected from about 0.125 mg to about 5 mg, and that amount is administered once per day.

7. A method according to claim 6 wherein the amount of 2-[(5-Chloro-2-{[3-methyl-1-(1-methylethyl)-1H-pyrazol-5-yl]amino}-4-pyridinyl)amino]-N-(methyloxy)benzamide hydrochloride, is selected from about 20 mg to about 500 mg, and that amount is administered once or twice per day; and the amount of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide is selected from about 0.125 mg to about 4 mg, and that amount is administered once per day; and
the combination is administered for a period of at least 14 consecutive days.

8. A method according to claim 7 wherein 2-[(5-Chloro-2-{[3-methyl-1-(1-methylethyl)-1H-pyrazol-5-yl]amino}-4-pyridinyl)amino]-N-(methyloxy)benzamide hydrochloride and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide, are administered within 12 hours of each other for from 1 to 3 consecutive days followed by administration of 2-[(5-Chloro-2-{[3-methyl-1-(1-methylethyl)-1H-pyrazol-5-yl]amino}-4-pyridinyl)amino]-N-(methyloxy)benzamide hydrochloride for from 3 to 7 consecutive days, optionally followed by one or more cycles of repeat dosing.

9. A method according to claim 5 where the cancer selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

10. A method according to claim 9 wherein the amount of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, is selected from about 10 mg to about 1,000 mg, and that amount is administered once or twice per day, and the amount of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 0.1 mg to about 5 mg, and that amount is administered once per day.

11. A method according to claim 10 wherein the amount of 52-[(5-Chloro-2-{[3-methyl-1-(1-methylethyl)-1H-pyrazol-5-yl]amino}-4-pyridinyl)amino]-N-(methyloxy)benzamide hydrochloride, is selected from about 20 mg to about 800 mg, and that amount is administered once or twice per day, and the amount of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide, is selected from about 0.125 mg to about 4 mg, and that amount is administered once per day, and the combination is administered for a period of at least 14 consecutive days.

12. A method according to claim 11 wherein 2-[(5-Chloro-2-{[3-methyl-1-(1-methylethyl)-1H-pyrazol-5-yl]amino}-4-pyridinyl)amino]-N-(methyloxy)benzamide hydrochloride and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide, are administered within 12 hours of each other for from 1 to 3 consecutive days followed by administration of 2-[(5-Chloro-2-{[3-methyl-1-(1-methylethyl)-1H-pyrazol-5-yl]amino}-4-pyridinyl)amino]-N-(methyloxy)benzamide for from 3 to 7 consecutive days, optionally followed by one or more cycles of repeat dosing.

13. A method according to claim 9 wherein the cancer selected from mesothelioma, lung, melanoma, glioblastoma, thyroid, breast, pancreatic, renal cell carcinoma, ovarian, head and neck, and endometrial.

14-16. (canceled)

17. A method treating a cancer selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer; in a human in need thereof which comprises the in vivo administration of a therapeutically effective amount of a combination of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, to such human, wherein the compounds of the combination are administered sequentially.

18. A method according to claim 17 wherein the amount of 52-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, is selected from about 10 mg to about 1,000 mg, and the amount of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 0.125 mg to about 5 mg.

19. A method according to claim 18 wherein the amount of 2-[(5-Chloro-2-{[3-methyl-1-(1-methylethyl)-1H-pyrazol-5-yl]amino}-4-pyridinyl)amino]-N-(methyloxy)benzamide, or a pharmaceutically acceptable salt thereof, is selected from about 20 mg to about 800 mg, and the amount of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof, is selected from about 0.125 mg to about 4 mg.

20. A method according to claim 19 wherein 2-[(5-Chloro-2-{[3-methyl-1-(1-methylethyl)-1H-pyrazol-5-yl]amino}-4-pyridinyl)amino]-N-(methyloxy)benzamide is administered for from 1 to 30 consecutive days, followed by an optional drug holiday of from 1 to 14 days, followed by administration of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide, for from 1 to 30 days, optionally followed by one or more cycles of repeat dosing.

21. A method according to claim 17 wherein the cancer selected from mesothelioma, lung, melanoma, glioblastoma, thyroid, breast, pancreatic, renal cell carcinoma, ovarian, head and neck, and endometrial.

22-24. (canceled)

25. A method according to claim 20 wherein 2-[(5-Chloro-{[3-methyl-1-(1-methylethyl)-1H-pyrazol-5-yl]amino}-4-pyridinyl)amino]-N-(methyloxy)benzamide is administered for from 1 to 21 consecutive days, followed by a drug holiday of from 3 to 10 days, followed by administration of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide for from 1 to 21 days, optionally followed by one or more cycles of repeat dosing.

26. (canceled)

27. A method according to claim 6 wherein 2-[(5-Chloro-{[3-methyl-1-(1-methylethyl)-1H-pyrazol-5-yl]amino}-4-pyridinyl)amino]-N-(methyloxy)benzamide and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide are administered within 12 hours of each other for 2 consecutive days followed by administration of 2-[(5-Chloro-{[3-methyl-1-(1-methylethyl)-1H-pyrazol-5-yl]amino}-4-pyridinyl)amino]-N-(methyloxy)benzamide for from 4 to 6 consecutive days, optionally followed by one or more cycles of repeat dosing.

28. A method according to claim 7 wherein 2-[(5-Chloro-{[3-methyl-1-(1-methylethyl)-1H-pyrazol-5-yl]amino}-4-pyridinyl)amino]-N-(methyloxy)benzamide and N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide are administered within 12 hours of each other for 2 days over a 7 day period, and during the other days of the 7 day period 2-[(5-Chloro-{[3-methyl-1-(1-methylethyl)-1H-pyrazol-5-yl]amino}-4-pyridinyl)amino]-N-(methyloxy)benzamide is administered alone, optionally followed by one or more cycles of repeat dosing.

29-31. (canceled)
